(19) United States
(12) Patent Application Publication
(10) Pub. No.: US 2006/0211063 A1
(43) Pub. Date: Sep. 21, 2006

(54) Phosphokinase assay

(75) Inventors: Jon M. Supernault, Ann Arbor, MI (US); Mark Cameron, Ann Arbor, MI (US); Sara Brien, Ann Arbor, MI (US)

Correspondence Address: Amy E Rinaldo;Kohn & Associates, Suite 410, 30500 Northwestern Highway, Farmington Hills, MI, 48334, US

(73) Assignee: ASSAY DESIGNS INC., Ann Arbor, MI (US)

(21) Appl. No.: 10/549,078

(22) PCT Filed: Mar. 12, 2004
(86) PCT No.: PCT/US04/07569

§ 371 Date: May 18, 2006

Related U.S. Application Data
(60) Provisional application No. 60/454,068, filed on Mar. 12, 2003.

(51) Int. Cl. G01N 33/53

(52) U.S. Cl. 435/7.92

(57) ABSTRACT

A method of detecting protein kinases in a sample by coating a solid surface with a buffer containing protease and phosphatase inhibitors and immobilizing a labeled antibody to the coated solid surface, whereby protein kinases react with the antibody, thereby detecting a presence of the protein kinases. Also provided is an assay and kit for detecting protein kinases having antibodies specific to a phosphorylation site of the protein kinase and a label bound to the antibodies.

### BACKGROUND OF THE INVENTION

1. Technical Field

The present invention relates to a phosphokinase assay. More specifically, the present invention relates to an immunometric phosphokinase assay.

2. Description of the Related Art

A phosphate tightly associated with a molecule, e.g., a protein, has been known since the late nineteenth century. Since then, a variety of covalent linkages of phosphate to proteins have been found. The most common linkages involve esterification of phosphate to serine, threonine, and tyrosine with smaller amounts being linked to lysine, arginine, histidine, aspartic acid, glutamic acid, and cysteine. The occurrence of phosphorylated molecules, e.g., proteins, implies the existence of one or more kinases, e.g., protein kinases, capable of phosphorylating various molecules, e.g., amino acid residues on proteins, and also of phosphatases, e.g., protein phosphatases, capable of hydrolyzing various phosphorylated molecules, e.g., phosphorylated amino acid residues on proteins.

Protein kinases play critical roles in the regulation of biochemical and morphological changes associated with cellular growth and division (D'Urso et al. (1990) Science 250:786-791; Birchmeier et al. (1993) Bioassays 15: 185-189). Protein kinases serve as growth factor receptors and signal transducers and have been implicated in cellular transformation and malignancy (Hunter et al. (1992) Cell 70:375-387; Posada et al. (1992) Mol. Biol. Cell 3:583-592; Hunter et al. (1994) Cell 79:573-582). For example, protein kinases have been shown to participate in the transmission of signals from growth-factor receptors (Sturgill et al. (1988) Nature 344:715-718; Gomez et al. (1991) Nature 353:170-173), control of entry of cells into mitosis (Nurse (1990) Nature 344:503-508; Mailer (1991) Curr. Opin. Cell Biol. 3:269-275) and regulation of actin bundling (Husain-Chishti et al. (1988) Nature 334:718-721).

Protein kinases can be divided into different groups based on either amino acid sequence similarity or specificity for either serine/threonine or tyrosine residues. A small number of dual-specificity kinases have also been described. Within the broad classification, kinases can be further subdivided into families whose members share a higher degree of catalytic domain amino acid sequence identity and also have similar biochemical properties. Most protein kinase family members also share structural features outside the kinase domain that reflect the kinase's particular cellular roles. Such roles include regulatory domains that control kinase activity or interaction with other proteins (Hanks et al. (1988) Science 241:42-52).

Extracellular-signal-regulated kinases/microtubule-associated protein kinases (Erk/MAPKs) and cyclin-directed kinases (Cdks) represent two large families of serine-threonine kinases (see Songyang et al., (1996) Mol. Cell. Biol. 16: 6486-6493). Both types of kinases function in cell growth, cell division, and cell differentiation in response to extracellular stimuli. The Erk/MAPK family members are critical participants in intracellular signaling pathways. Upstream activators, as well as the Erk/MAPK components, are phosphorylated following contact of cells with growth factors or hormones or after cellular stressors, for example, heat, ultraviolet light, and inflammatory cytokines. The kinases transport messages that have been relayed from the plasma membrane to the cytoplasm, by upstream kinases, into the nucleus where the kinases phosphorylate transcription factors and effect gene transcription modulation (Karin et al., (1995) Curr. Biol. 5: 747-757). Substrates of the Erk/MAPK family include c-fos, c-jun, APF2, and ETS family members Elk1, Sap1a, and c-Ets-1 (cited in Brott et al., (1998) Proc. Natl. Acad. Sci. USA 95, 963-968).

Cdks regulate transitions between successive stages of the cell cycle. The activity of the molecules is controlled by phosphorylation events and by association with cyclin. Cdk activity is negatively regulated by the association of small inhibitory molecules (Dynlacht, (1997) Nature 389:148-152). Cdk targets include various transcriptional activators such as p110Rb, p107 and transcription factors, such as p53, E2F and RNA polymerase II, as well as various cytoskeletal proteins and cytoplasmic signaling proteins (cited in Brott et al., above).

The Extracellular signal-Related Kinase (ERK) is expressed in two molecular weight forms, one at 42 kilodalton (ERK2) and one at 44 kilodalton (ERK1). Both Erk1 and Erk<2 function in a protein kinase cascade that plays a critical role in the regulation of cell growth and differentiation. ERK is activated by a wide variety of extracellular signals including growth and neurotrophic factors, cytokines, hormones and neurotransmitters. Activation of the ERK kinases occurs through phosphorylation of threonine and tyrosine at the sequence T*EY* by another kinase.

A protein has been isolated in Drosophila, designated nemo, which has homology to Erk/MAPKs and Cdks. A mammalian homologue of nemo, designated NLK, has also been reported (Brott et al., above). The protein kinase autophosphorylates and localizes to a great extent in the nucleus. The protein showed homology to both families of kinases (Erk/MAPKs and Cdks). It did not possess the characteristic MAPK phosphorylation motif TXY in the conserved kinase domain vm. It instead exhibited the sequence TQE resembling the THE sequence found in some Cdks.

Typical methods for detecting the presence of phosphorylated ERK involve the use of Western blotting in which the proteins in cell lysates are separated on an SDS-PAGE gel. The proteins are then transferred to a nitrocellulose membrane and the presence of a specific protein is detected using an antibody. For example, in a Western blot for ERK, the transferred proteins immobilized onto the nitrocellulose membrane would be detected by using an antibody specific for ERK1 and/or ERK2. The specific antibody binds to any ERK on the membrane and the presence of the specific antibody is detected after washing the membrane with another antibody-enzyme conjugate. The presence of the antibody-enzyme conjugate is detected after washing the membrane again using a substrate, typically a chemiluminescent substrate that produces a visible band on photographic film.

The presence of the phosphorylated ERK protein can be estimated by assessing the level of staining of the bands produced on the photographic film. The estimation can be quite accurate if all the variables of the SDS-PAGE electrophoresis transfer to the nitrocellulose membrane, specific antibody binding, generic antibody-enzyme conjugate binding and substrate steps can be controlled. In many instances there are minor problems with one or more steps that make precision difficult in the long and laborious process (the process can take 36 hours or more to complete). A major problem with detecting and quantifying all post-translational modified proteins is that the modified protein can be converted into another form. For example phosphatase enzymes are present in many samples and are a major source of contamination. The enzymes will cleave phosphate groups off of the target phosphokinases and so prevent the accurate quantitation of the protein.

The most widely used technique for measuring protein kinase activity is based on radioactive detection. In the method, a sample containing the kinase of interest is incubated with activators and a substrate in the presence of gamma ³²P-ATP. After a suitable incubation period, the reaction is stopped and an aliquot of the reaction mixture is placed directly onto a filter that binds the substrate. The filter is then washed multiple times to remove excess radioactivity, and the amount of radiolabeled phosphate incorporated into the substrate is measured by scintillation counting.

The above method is widely used and provides one method for determining protein kinase activity in both crude and purified samples. However, because of the necessity of multiple washings, which are generally done by manually transferring the filter to a beaker and washing and rinsing with gentle agitation, the procedure is quite time consuming.

Other methods for detecting kinase activity are based on separations due to the charge differences between phosphorylated and non-phosphorylated proteins and peptides. Techniques based on gel electrophoresis and HPLC have, among others, been used. In combination with the above techniques, spectrophotometric and fluorometric detection have also been used. International Patent Application WO 93/10461 and U.S. Pat. Nos. 5,120,644 and 5,141,852, to Ikenaka, et al. describe many methods heretofore used for detecting protein kinase activity. Also reference is directed to Analytical Biochemistry, 209, 348-353, 1993, “Protein Kinase Assay Using Tritiated Peptide Substrates and Ferric Adsorbent Paper for Phosphopeptide Binding.” that teaches additional methods of separation.

There are several additional approaches to analyzing the state of modification of target proteins in vivo. Such methods include, but are not limited to, in vivo labeling of cellular substrate pools with radioactive substrate or substrate precursor molecules to result in incorporation of labeled (for example, radiolabeled) moieties (e.g., phosphate, fatty acyl (including, but not limited to, myristoyl, palmityl, sentrin, methyl, actyl, hydroxyl, iodine, flavin, ubiquitin or ADP-ribosyls), which are added to target proteins. Analysis of modified proteins is typically performed by electrophoresis and autoradiography, with specificity enhanced by immunoprecipitation of proteins of interest prior to electrophoresis. Another method is back-labeling, involving the enzymatic incorporation of a labeled (including, but not limited to, with a radioactive and fluorescent label) moiety into a protein in vitro to estimate the state of modification in vivo. The detection of alteration in electrophoretic mobility of modified protein compared with unmodified (e.g., glycosylated or ubiquitinated) protein can also be used. Gel-shift analysis of radiolabeled oligonucleotides binding to modified proteins, thin-layer chromatography of radiolabeled fatty acids extracted from the protein of interest, partitioning of protein into detergent-rich or detergent layer by phase separation, and the effects of enzyme treatment of the protein of interest on the partitioning between aqueous and detergent-rich environments can also be used.

The use of cell-membrane-permeable protein-modifying enzyme inhibitors (e.g., Wortmannin, staurosporine) to block modification of target proteins and comparable inhibitors of the enzymes involved in other forms of protein modification (above) can be used as well as antibody recognition of the modified form of the protein (e.g., using an antibody directed at ubiquitin or carbohydrate epitopes), e.g., by Western blotting, of either 1- or 2-dimensional gels bearing test protein samples. Lectin-protein interaction in Western blot format can be used as an assay of the presence of particular carbohydrate groups (defined by the specificity of the lectin in use). The exploitation of eukaryotic microbial systems to identify mutations in protein-modifying enzymes is another method of detection.

All of the above strategies have certain limitations. Monitoring states of modification by pulse or steady state labeling is merely a descriptive strategy to show which proteins are modified when samples are separated by gel electrophoresis and visualized by autoradiography. The result is unsatisfactory due to the inability to identify many of the proteins that are modified. A degree of specificity is afforded by the technique if it is combined with immunoprecipitation; however, the technique is of course limited by the availability of antibodies to target proteins. Moreover, only highly expressed proteins are readily detectable using the technique, which may fail to identify many low-abundance proteins, which are potentially important regulators of cellular functions.

The use of enzyme inhibitors to block activity is also thought to be problematic. For example, very few kinase inhibitors have adequate specificity to allow for the unequivocal correlation of a given kinase with a specific kinase reaction. Indeed, many inhibitors have a broad inhibitory range. For example, staurosporine is a potent inhibitor of phospholipid/Ca⁺2 dependant kinases. Wortmannin is somewhat more specific, being limited to the phosphatidylinositol-3 kinase family. The result is unsatisfactory because more than one biochemical pathway may be affected during treatment making the assignment of the effects almost impossible.

Yeast (Saccharomyces cervisiae and Schizosaccharomyces pombe) has been exploited as a model organism for the identification of gene function using recessive mutations. It is through research on the effects of the mutations that the functional specificities of many protein-modifying enzymes have been elucidated. However, the molecular genetic techniques are not easily transferable to higher eukaryotes, which are diploid and therefore not as genetically tractable as the lower eukaryotes.

While there is disclosure in the prior art for methods of detecting protein kinases in a sample, there is no disclosure for a precise method of detecting small quantities of protein kinase in a sample. It would therefore be useful to develop a more precise method of detecting protein kinase in a sample.

### SUMMARY OF THE INVENTION

According to the present invention, there is provided a method of detecting protein kinases in a sample by coating a solid surface with an excess of a kinase specific antibody in a buffer containing protease and phosphatase inhibitors. This solid phase antibody acts as immunoaffinity isolation medium whereby protein kinases react with the antibody, thereby allowing the kinase to be isolated from other similar molecules present in the sample. Also provided are antibodies specific to a phosphorylation site of the protein kinase and a method for detecting such phosphorylation specific antibodies.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a graph depicting a pERK typical standard curve;

FIG. 2 is a graph depicting a pERK plate test;

FIG. 3 is a graph depicting the standard day-to-day variation of concentration;

FIG. 4 is a graph depicting the standard variation of concentration between different shipments from the same lot;

FIG. 5 is a graph depicting the antibody reproducibility between different runs;

FIG. 6 is a graph depicting the antibody reproducibility between different individuals;

FIG. 7 is a graph depicting the stability of the assays at different temperatures (i.e. freeze/thaw data); and

FIG. 8 is a graph depicting the stability of the assays at low temperatures for extended periods of time.

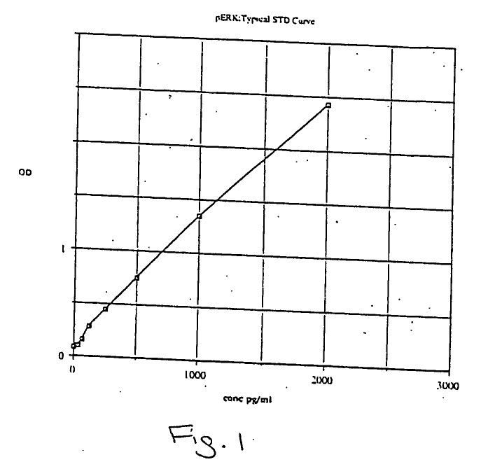

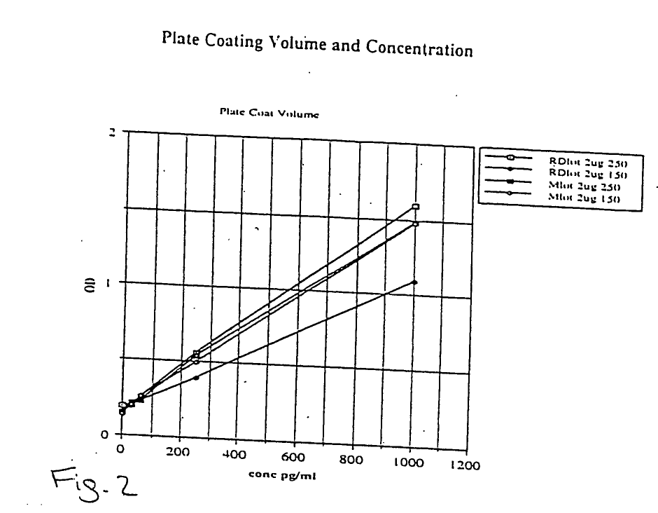

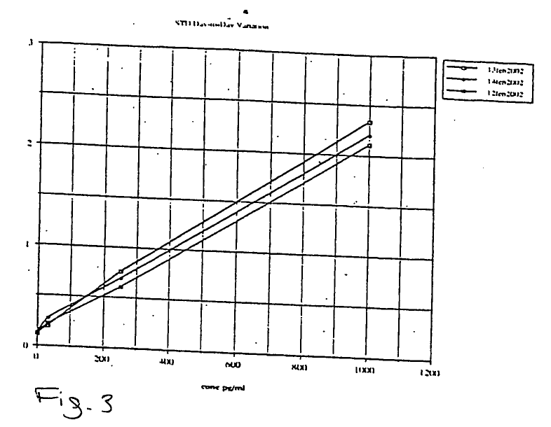

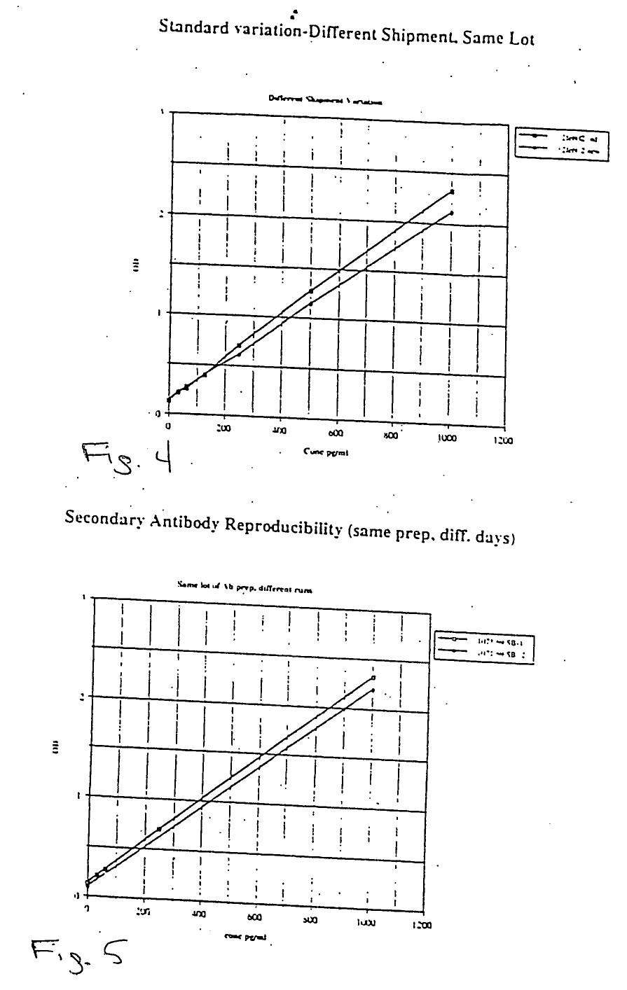

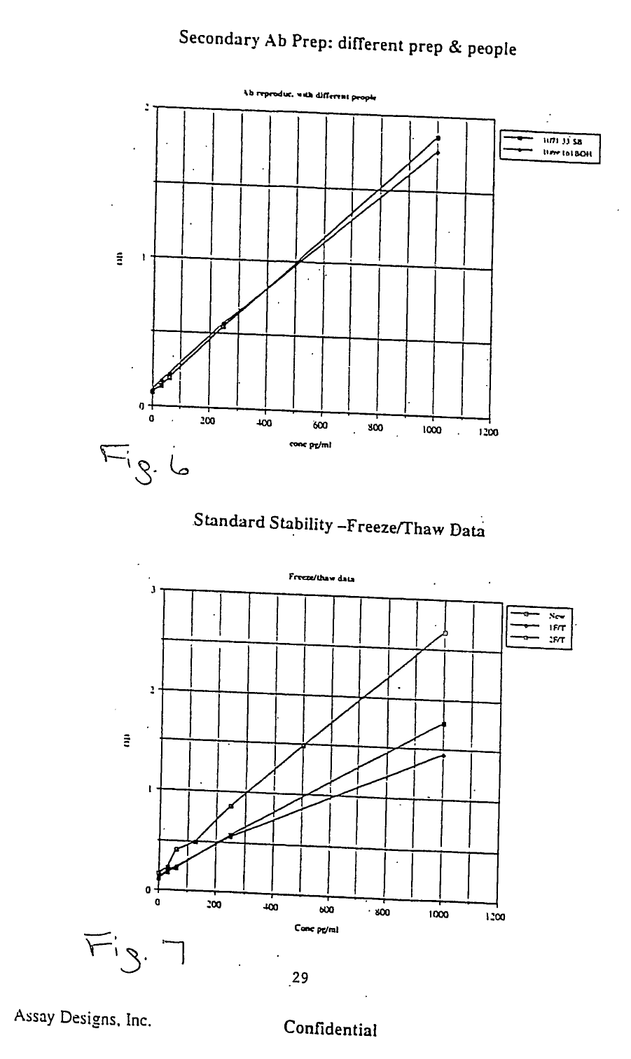

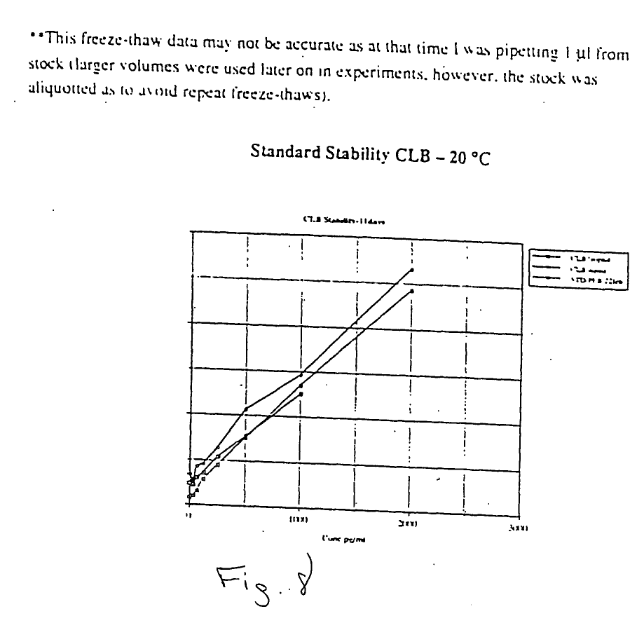

### DESCRIPTION OF THE INVENTION

Generally, the present invention provides a method of preserving the integrity of the phosphokinase molecule in a phosphokinase assay that enables the precise quantitation of a protein kinase. More specifically, the present invention by inhibiting the action of phosphokinase degrading enzymes provides an assay for accurately detecting small quantities of protein kinase in a sample.

The term “protein kinase” as used herein is intended to include, but is not limited to, enzymes that covalently modify proteins and peptides by the attachment of a phosphate group to one or more sites on the protein or peptide. For example, the enzyme can transfer the phosphoric acid at the gamma-position of ATP to a hydroxyl group of serine, threonine, tyrosine and so forth, and can play the role of a central mechanism in the transmission of information into cells. Protein kinases play an important role in the function regulatory mechanisms of almost all cells. Protein kinases have been clearly shown to be involved in cell movement, cell growth, metabolic response, immune response and so forth.

The term “phosphorylation” is defined as a well-studied example of an enzymatic modification of a protein. There are many cases in which polypeptides form higher order tertiary structures with like polypeptides (homo-oligomers) or with unlike polypeptides (hetero-oligomers). In the simplest scenario, two identical polypeptides associate to form an active homodimer. An example of one type of association is the natural association of myosin H molecules in the assembly of myosin into filaments.

The term “label” as used herein is intended to include any product that can be attached to a compound or antibody that is able to be measured. Examples of such labels include, but are not limited to, fluorescent labels, luminescent labels, chromogenic molecules, radioactive tags, and especially enzymes capable of generating such signals and other similar labels known to those of skill in the art.

The “inhibitors” that can be added include, but are not limited to, ethylenediamine tetraacetic acid (EDTA) and its analog EGTA that chelate calcium ions (activators of some proteases), β-glycerophosphate and activated sodium orthovanadate that inhibit phosphatase activity, phenylmethyl sulfonylfluoride (a serine and cysteine protease inhibitor) along with commercially available protease inhibition cocktails to inhibit a variety of proteases.

The assay of the present invention can be performed on any solid phase surface and in any configuration of such solid phase. For example, the assay can be performed in multiple well plates, single well plates, on membranes, on microparticles or other solid phase. Examples of solid phases that can be used are known to those of skill in the art.

The antibody is a very specific antibody and can be made using techniques known to those of skill in the art. The specificity of the antibody refers not only to the specific protein sequence, but also to the conformation of the protein. The specificity of the antibody enables the assay to be more precise and to detect small amounts of the protein in a sample. Further, the antibody can also be specific to another aspect of a cascade of which the protein is part. For example, the antibody can be specific to the phosphorylation sequence in ERK1 and ERK2 related to threonine and tyrosine at the sequence T*EY*. This sequence is phosphorylated by an upstream kinase to activate ERK through attachment of phosphate groups to the threonine and tyrosine residues. Antibodies raised to the sequence corresponding to residues 181 to 191 for ERK2, TGFL(p)TE(p)YVATR bind only to the phosphoERK molecule.

The assay also includes a buffer that maximizes the integrity of the protein kinase. Some of the buffers must also be capable of lysing cells. However, the lysing of the cells must be accomplished by disrupting the cell membranes without affecting the structure and function of the protein. In the preferred embodiment, the buffer is an SDS lysis buffer, however other buffers possessing the capabilities that are known to those of skill in the art can also be utilized in conjunction with the assay of the present invention.

The assay of the present invention is able to detect very minute amounts of protein kinase in a sample. For example, the assay is able to detect picograms of protein kinase in a sample. The assay is able to function in the above manner because the assay utilizes a specific antibody to the phosphorylation site for detecting the protein kinase. More specifically, the present invention provides an assay for the detection of a protein that functions by attaching an antibody, specific to the phosphorylation site of the protein being detected, to a surface of a vessel that is capable of performing the assay and is used for conducting the assay. The assay can be performed on any solid phase surface.

Specifically, the methods used in the production of the assay and kit of the present invention functions as follows. The assay and kit require special plate coating procedures to be followed in order to minimize the presence of endogenous protease and phosphatases enzymes that can degrade the integrity of the analyte being tested. In a typical plate coating process a monoclonal antibody to ERK1 and 2 is dissolved in a phosphate buffer and applied to the microtiter plate. In order not to coat the plate with endogenous proteases and phosphatases or any other degraditive enzyme that might be present on the plastic well, special additives can be added either to the monoclonal preparation or in the coating buffers. Activated sodium orthovanadate is a known irreversible inhibitor of phosphatases and the addition of the material, along with other inhibitors of degradative activity, is essential in order to protect the sample and standard added to the solid phase during testing.

Preferably, a cocktail of chemicals and biochemicals are added to the components of the assay to inhibit phosphatase and protease activity. Such chemicals can be added to the chemicals used for coating and blocking plates, for the buffers used for dilution of the samples, and for the buffers used in preparation of the standards used in the kit.

In a typical embodiment the monoclonal antibody is dissolved in a typical coating buffer modified to contain a low concentration of calcium chelators such as EDTA, known to inhibit some calcium activated proteases and a phosphatase inhibitor β-glycerophosphate, known to inhibit phosphatases. The antibody solution is allowed to interact with the plastic solid phase surface overnight and is then aspirated off. Instead of blocking the plate surfaces with a BSA protein, which can contain enzymes that degrade phosphokinases, the plate is blocked with a special blocking buffer in order to protect the kinase samples. The blocking buffer contains a PBS buffer with detergents added to block the plate surface and a phosphatase inhibitor activated sodium orthovanadate. Because of the lack of stability of activated orthovanadate above −20° C. the phosphatase inhibitor can be added to the blocking buffer immediately prior to blocking the plate surfaces to irreversibly inactivate phosphatases in the buffers, antibody, or on the solid phase.

Other embodiments include the addition of calcium chelators, EDTA (to inhibit metalloproteases and calcium activated proteases) and β-glycerophosphate (to inhibit phosphatases) to the buffers used for coating and blocking the ERK monoclonal onto the microtiter plate. In a typical assay using a 2,000 pg/mL phospho-ERK (pERK) standard the optical density increased from 1.7 to 2.4 indicating the inhibition of protease/phosphatase activity and preservation of the phosphokinase after addition of phosphatase and protease inhibitors.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

General Methods:

General methods in molecular biology: Standard molecular biology techniques known in the art and not specifically described are generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

General methods in immunology: Standard methods in immunology known in the art and not specifically described are generally followed as in Stites et al. (eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

Immunoassays

In general, ELISAs are the preferred immunoassays employed to assess a specimen. ELISA assays are well known to those skilled in the art. Both polyclonal and monoclonal antibodies can be used in the assays. Where appropriate, other immunoassays, such as radioimmunoassays (RIA), can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, Molecular Cloning: A Laboratory Manual, Cold Springs Harbor, N.Y., 1989

Antibody Production

Antibody Production: Antibodies can be either monoclonal, polyclonal or recombinant. Conveniently, the antibodies can be prepared against the immunogen or portion thereof. For example, a synthetic peptide based on the sequence, or prepared recombinantly by cloning techniques or the natural gene product and/or portions thereof can be isolated and used as the immunogen. Immunogens can be used to produce antibodies by standard antibody production technology well known to those skilled in the art as described generally in Harlow and Lane, Antibodies: A Laboratory Manual, Cold. Spring Harbor Laboratory, Cold Spring Harbor, N.Y., 1988 and Borrebaeck, Antibody Engineering—A Practical Guide, W.H. Freeman and Co., 1992. Antibody fragments also can be prepared from the antibodies and include Fab, F(ab′)₂, and Fv by methods known to those skilled in the art.

For producing polyclonal antibodies, a host, such as a rabbit or goat, is immunized with the immunogen or immunogen fragment, generally with an adjuvant, and if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera, rendering it monospecific.

For producing monoclonal antibodies, the technique involves hyperimmunization of an appropriate donor with the immunogen, generally a mouse, and isolation of splenic antibody producing cells. The cells are fused to a cell having immortality, such as a myeloma cell, to provide a fused cell hybrid that has immortality and secretes the required antibody. The cells are then cultured, in bulk, and the monoclonal antibodies harvested from the culture media for use.

For producing recombinant antibody (see generally Huston et al, 1991; Johnson and Bird, 1991; Mernaugh and Mernaugh, 1995), messenger RNAs from antibody producing B-lymphocytes of animals, or hybridoma, are reverse-transcribed to obtain complimentary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. (For a general discussion of conjugation of fluorescent or enzymatic moieties see Johnstone & Thorpe, Immunochemistry in Practice, Blackwell Scientific Publications, Oxford, 1982). The binding of antibodies to a solid support substrate is also well known in the art. (For a general discussion, see Harlow & Lane Antibodies: A Laboratory Manual, Cold Spring Harbor Laboratory Publications, New York, 1988 and Borrebaeck, Antibody Engineering—A Practical Guide, W.H. Freeman and Co., 1992) The detectable moieties contemplated with the present invention can include, but are not limited to, fluorescent, metallic, enzymatic and radioactive markers such as biotin, gold, ferritin, alkaline phosphatase, b-galactosidase, peroxidase, urease, fluorescein, rhodamine, tritium, ¹⁴C and iodination.

### Example 1

In order to allow faster and more precise measurement of such molecules in large numbers of samples, methods that allow the transient phosphorylated proteins to be detected and quantified accurately needed to be developed. The easiest way to accomplish the effect was to develop an ELISA method for phosphorylated proteins. ELISA's have been used in both clinical and research applications for a number of years, but the measurement of phosphorylated proteins required significant modifications.

Most typical ELISA products are designed to measure stable non-modified proteins. An example is the ELISA for human interleukin 6 (hlL-6). In the kit no special precautions are needed in the preparation of the components of the kit or with the samples to be tested in order to protect the hlL-6 in the standard or samples, as the hlL-6 is stable for significant periods of time. In the preparation of the hlL-6 kit a monoclonal antibody to hlL-6 is bound to the microtiter plate by dissolving the antibody in a PBS buffer containing just a preservative. After the solution has stood at room temperature overnight the antibody solution is aspirated and a PBS buffer containing a high concentration of bovine serum albumin (BSA) is added. The BSA coats any active surfaces on the plate to ensure that proteins in the sample do not non-specifically bind to the plate surface. However proteins such as BSA can contain very high levels of phosphatases and other protein modifying enzymes as contaminants.

Specifically, Applicant's created a phosphoERK1/2 ELISA kit. The kit requires special plate coating procedures to be followed to minimize the presence of endogenous phosphatases. In a typical plate coating process a monoclonal antibody to ERK1 and 2 is dissolved in a phosphate buffer and applied to the microtiter plate. In order not to coat the plate with endogenous phosphatases that might be present on the plastic well, special additives can be added either to the monoclonal preparation or in the coating buffers. Activated sodium orthovanadate is a known inhibitor of phosphatases and the addition of the material, along with other inhibitors of phosphatase activity, is beneficial in order to protect the sample and standard added to the plate during testing.

It is impossible to accurately determine the levels of phosphoproteins without the addition of a cocktail of chemicals and biochemicals to inhibit phosphatase and protease activity. Such chemicals can be added to the buffers used for coating and blocking plates, for the buffers used for dilution of the samples, and for the buffers used in preparation of the standards used in the kit.

In a typical embodiment the monoclonal antibody is dissolved in a typical coating buffer modified to contain a low concentration of calcium chelators such as EDTA, known to inhibit some calcium activated proteases and a phosphatase inhibitor β-glycerophosphate, known to inhibit phosphatases. The antibody solution is allowed to interact with the plastic solid phase surface overnight and is then aspirated off. Instead of blocking the plate surfaces with a BSA protein containing solution a special blocking buffer is utilized in order to protect the kinase samples. The blocking buffer contains a PBS buffer with detergents added to block the plate surface and a phosphatase inhibitor activated sodium orthovanadate. Because of the lack of stability of activated orthovanadate above −20° C. the phosphatase inhibitor can be added to the blocking buffer immediately prior to blocking the plate surfaces.

After the addition of both the calcium chelators, EDTA (to inhibit metalloproteases and calcium activated proteases) and β-glycerophosphate (to inhibit phosphatases) to the buffers used for coating and blocking the ERK monoclonal onto the microtiter plate, the optical density of the 2,000 pg/mL phospho-ERK (pERK) standard increased from 1.7 to 2.4 indicating the inhibition of protease/phosphatase activity and preservation of the phosphokinase.

In a similar experiment a monoclonal antibody is coated onto microtiter plates to create a phospho-p38 ELISA kit (p38 MAP kinase or mitogen-activated protein kinase 14, MAP kinase p38). The addition of activated sodium orthovanadate (a potent phosphatase inhibitor) to the blocking buffer (a PBS based buffer containing detergents and EDTA, to inhibit calcium-activated proteases) and β-glycerophosphate (to inhibit phosphatases)) increased the signal for the standard from 2.6 optical density units bound to 3.1. The signal to noise ratio for the 5,000 pg/mL standard compared to the 0 pg/mL standard increased from 37.1 to 44.3 with no change in the 0 pg/mL standard signal. The increase indicated that binding of the phospho p38 standard to the coated solid phase well is protected by the addition of activated sodium orthovanadate to the plate coating and blocking buffers.

The addition of the typical inhibitors to assay buffers allows for the endogenous kinases, phosphokinases, phosphatases and other proteins to be protected during the binding step to the monoclonal capture antibody. The inhibitors that can be added include, but are not limited to, ethylenediamine tetraacetic acid (EDTA) and its analog EGTA that chelate calcium ions (activators of some proteases), β-glycerophosphate and activated sodium orthovanadate that inhibit phosphatase activity, phenylmethyl sulfonylfluoride (a serine and cysteine protease inhibitor) along with commercially available protease inhibition cocktails to inhibit a variety of proteases.

In conclusion, there has been demonstrated that in order to accurately detect the presence of kinases it is a beneficial to add certain inhibitors of proteases and phosphatases to protect the integrity of the protein and the phosphate groups attached to the kinases.

### Example 2

Applicants created a phospho-Extracellular signal-Regulated Kinase (pERK) ½ TiterZyme® Enzyme Immunometric Assay (EIA) kit. The kit is a complete kit for the quantitative determination of pERK ½ in cell lysates. The kit uses a monoclonal antibody to ERK immobilized on a microtiter plate to bind pERK in the standards or sample. A recombinant pERK Standard is provided in the kit. After a short incubation the excess sample or standard is washed out and a rabbit polyclonal antibody to pERK is added. The antibody binds to the pERK captured on the plate. After a short incubation period, the excess antibody is washed out and donkey anti-rabbit IgG conjugated to Horseradish peroxidase is added, which binds to the polyclonal pERK antibody. Excess conjugate is washed out and substrate is added. After a short incubation period, the enzyme reaction is stopped and the color generated is read at 450 nm. The measured optical density is directly proportional to the concentration of pERK in either standards or samples.

The assay is compatible with pERK samples in a wide range of cell lysis matrices and buffers. Typical lysis buffers contain Tris or HEPES buffered saline at an approximate pH of 7.4 containing high concentrations (0.1 to 1%) of detergents (Triton X-100 or SDS) with added protease inhibitors, such as leupeptin and aprotinin, and with phosphatase inhibitors, such as β-glycerophosphate and sodium orthovanadate.

It is recommended that all samples be lysed with a cell lysis buffer. One such buffer is 50 mM β-glycerophosphate, 10 mM HEPES, pH 7.4, 70 mM NaCl, 2 mM EDTA, 1% SDS. Samples lysed in the cell lysis buffer must be diluted at least 1:100 in buffer prior to analysis. Alternatively, if samples are lysed in a buffer containing 1% Triton X-100, the samples must be diluted at least 1:1,000 in buffer prior to analysis.

Assay Procedure

All reagents were brought to room temperature for at least 30 minutes prior to opening. All standards, controls and samples can be run in duplicate.

The assay can be performed as follows. Pipet 100 μL of buffer into the wells. Pipet 100 μL of Standards into appropriate wells. Pipet 100 μL of the Samples into the appropriate wells. Tap the plate gently to mix the contents. Seal the plate and incubate at room temperature on a plate shaker for 1 hour at ˜500 rpm. Empty the contents of the wells and wash by adding 400 μL of wash solution to every well. Repeat the wash 3 more times for a total of 4 washes. After the final wash, empty or aspirate the wells and firmly tap the plate on a lint free paper towel to remove any remaining-wash buffer. Pipet 100 μL of yellow Antibody that reacts with a specific phosphorylated sequence of ERK into each well, except the Blank. Seal the plate and incubate at room temperature on a plate shaker for 1 hour at ˜500 rpm. Empty the contents of the wells and wash by adding 400 μL of wash solution to every well. Repeat the wash 3 more times for a total of 4 washes. After the final wash, empty or aspirate the wells and firmly tap the plate on a lint free paper towel to remove any remaining wash buffer. Add 100 μL of blue Conjugate that reacts with the polyclonal antibody to pERK bound to the pERK bound to the plate to each well, except the Blank. Seal the plate and incubate at room temperature on a plate shaker for 30 minutes at ˜500 rpm. Empty the contents of the wells and wash by adding 400 μL of wash solution to every well. Repeat the wash 3 more times for a total of 4 washes. After the final wash, empty or aspirate the wells and firmly tap the plate on a lint free paper towel to remove any remaining wash buffer. Pipet 100 μL of Substrate Solution into each well. Incubate for 15 minutes at room temperature on a plate shaker at ˜500 rpm. Pipet 100 μL of Stop Solution to each well. Blank the plate reader-against the Blank wells, read the optical density at 450 nm, preferably with correction between 570 and 590 nm. If the plate reader is not able to be blanked against the Blank wells, manually subtract the mean optical density of the Blank wells from all the readings.

| | | | Perk |
|---|---|---|---|
| Sample | Average OD | Net OD | (pg/mL) |
| Blank | (0.091) | | |
| S0 | 0.0.191 | 0.100 | 0 |
| S1 | 2.795 | 1.1162.704 | 2,000 |
| S2 | 1.398 | 0.5791.307 | 1,000 |
| S3 | 0.734 | 0.3030.643 | 500 |
| S4 | 0.440 | 0.1690.349 | 250 |
| S5 | 0.306 | 0.1080.215 | 125 |
| S6 | 0.250 | 0.0710.159 | 62.5 |

Performance Characteristics

The following parameters for the kit were determined using the guidelines listed in the National Committee for Clinical Laboratory Standards (NCCLS) Evaluation Protocols¹².

Sensitivity

Sensitivity was calculated by determining the average optical density bound for sixteen (16) wells run with 0 pg/mL Standard, and comparing to the average optical density for sixteen (16) wells run with a Standard. The detection limit was determined as the concentration of pERK measured at two (2) standard deviations from the 0 pg/mL. Sensitivity was calculated as 38.3 pg/mL.

Linearity

A sample containing 1,248.7 pg/mL pERK was serially diluted 4 times 1:2 in the buffer supplied in the kit and measured in the assay. The data was plotted graphically as actual pERK concentration versus measured pERK concentration.

The line obtained had a slope of 0.9513 with a correlation coefficient of 0.99730.9999.

Precision

Intra-assay precision was determined by taking samples containing low, medium and high concentrations of pERK and running the samples multiple times (n=I6) in the same assay. Inter-assay precision was determined by measuring two samples with low and high concentrations of pERK in multiple assays (n=8).

The precision numbers listed below represent the percent coefficient of variation for the concentrations of pERK determined in the assays as calculated by a four parameter logistic curve-fitting program.

| | pERK | | Inter-assay |
|---|---|---|---|
| | (pg/mL) | Intra-assay % CV | % CV |
| Low | 302.0 | 5.8 | |
| Medium | 882.3 | 6.4 | |
| High | 1,597.5 | 4.3 | |
| Low | 612.05 | | 11.5 |
| High | 1,151,.9 | | 4.9 |

Cross Reactivities

The pERK TiterZyme® EIA kit is specific for bioactive pERK. There is 0.5% cross-reactivity with p38. There is less than 0.01% cross-reactivity with non-phosphorylated ERK and JNK.

Sample Recoveries

pERK concentrations were measured in assay buffer after dilution from RIPA lysis buffer. Samples had to be diluted at least 1 part in 4 of assay buffer to achieve recoveries of 105.5%

Throughout the application, various publications, including United States patents, are referenced by author and year, and patents, by number. Full citations for the publications are listed below. The disclosures of the publications and patents in their entireties are hereby incorporated by reference into the application in order to more fully describe the state of the art to which the invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

1. An assay for detecting protein kinases, said assay comprising:
antibodies specific to a phosphorylation site of a protein kinase; and
label bound to said antibodies.

2. The assay according to claim 1, further including detecting means for detecting a presence of said label bound to said antibodies when said antibody is bound to the protein kinase.

3. The assay according to claim 2, wherein said label is a product capable of being detected by said detecting means.

4. The assay according to claim 3, wherein said label is selected from the group consisting essentially of fluorescent labels, luminescent labels, chromogenic molecules, and radioactive tags.

5. The assay according to claim 1, further including an inhibitor of protease and phosphatase activity.

6. The assay according to claim 5, wherein said inhibitors inhibit protease activities selected from the group consisting essentially of serine and cysteine protease activity.

7. The assay according to claim 5, wherein said inhibitor is selected from the group consisting essentially of ethylenediamine tetraacetic acid (EDTA), EGTA, β-glycerophosphate, activated sodium orthovanadate, phenylmethyl sulfonylfluoride, and protease inhibition cocktails.

8. The assay according to claim 2, wherein said detecting means is a detector capable of detecting visual labels.

9. The assay according to claim 2, wherein said detecting means is capable of detecting labels selected from the group consisting essentially of optical labels, fluorescent labels, and radioactive labels.

10. A kit for detecting protein kinases, said kit comprising:
antibodies specific to a phosphorylation site of a protein kinase; and
label bound to said antibodies.

11. The kit according to claim 10, further including detecting means for detecting a presence of said label bound to said antibodies when said antibodies are bound to the protein kinase.

12. The kit according to claim 11, wherein said label is a product capable of being detected by said detecting means.

13. The kit according to claim 12, wherein said label is selected from the group consisting essentially of fluorescent labels, luminescent labels, chromogenic molecules, and radioactive tags.

14. The kit according to claim 10, further including an inhibitor of protease and phosphatase activity.

15. The kit according to claim 14, wherein said inhibitors inhibit protease activities selected from the group consisting essentially of serine and cysteine protease activity.

16. The kit according to claim 14, wherein said inhibitor is selected from the group consisting essentially of ethylenediamine tetraacetic acid (EDTA), EGTA, β-glycerophosphate, activated sodium orthovanadate, phenylmethyl sulfonylfluoride, and protease inhibition cocktails.

17. The kit according to claim 10, wherein said detecting means is a detector capable of detecting visual labels.

18. The kit according to claim 10, wherein said detecting means is capable of detecting labels selected from the group consisting essentially of optical labels, fluorescent labels, and radioactive labels.

19. A method of detecting protein kinases in a sample by:
coating a solid surface with a buffer containing protease and phosphatase inhibitors;
immobilizing a labeled antibody to the coated solid surface, whereby protein kinases react with the antibody thereby detecting a presence of the protein kinases.

20. The method according to claim 19, further including detecting the presence of protein kinases in the sample.

21. A stable buffer comprising a buffer and protease inhibitors in said buffer.
